(19) United States
(12) Patent Application Publication
(10) Pub. No.: US 2020/0000873 A1
(43) Pub. Date: Jan. 2, 2020

(54) METHODS FOR IMPROVING THE COGNITIVE FUNCTIONS OF A SUBJECT

(71) Applicant: The Regents of the University of California, Oakland, CA (US)

(72) Inventors: David Feifel, La Jolla, CA (US); Jared Young, La Jolla, CA (US)

(21) Appl. No.: 16/480,968

(22) PCT Filed: Jan. 31, 2018
(86) PCT No.: PCT/US2018/016229

§ 371 Date: Jul. 25, 2019

Related U.S. Application Data
(60) Provisional application No. 62/453,458, filed on Feb. 1, 2017.

(51) Int. Cl. A61K 38/095; A61K 9/00; A61P 25/28

(52) U.S. Cl. CPC A61K 38/095; A61K 45/06; A61P 25/28; A61K 9/0043

(57) ABSTRACT

The disclosure provides for methods to improve the executive function of a subject by administering an effective amount of an oxytocin peptide, analog or mimetic.

### CROSS REFERENCE TO RELATED APPLICATIONS

This application claims priority under 35 U.S.C. § 119 to U.S. Provisional Application Ser. No. 62/453,458, filed Feb. 1, 2017. The disclosures of which are incorporated herein by reference.

### GOVERNMENT LICENSE RIGHTS

This invention was made with Government support under Grant No. RO1 MH103421-03, awarded by the National Institutes of Health. The Government has certain rights in the invention.

### TECHNICAL FIELD

Provided herein are methods to improve the cognitive function of a subject by administering an effective amount of an oxytocin peptide.

### BACKGROUND

Oxytocin is a human peptide hormone and neuropeptide that is produced by the hypothalamus and released by the posterior pituitary. Oxytocin has been implicated in the regulation of social functions including enhancing social affiliation and social cognition (understanding emotions or other members of ones species).

### SUMMARY

The disclosure provides for methods to improve the executive functions of a subject by administering an effective amount of an oxytocin peptide or a pharmaceutical composition thereof. In particular, it was found that the administration of oxytocin significantly improved the probabilistic learning of subjects.

The disclosure provides a method of treating a subject that has an impairment in executive functioning or prophylactically preventing an impairment in executive function of a subject comprising, administering to the subject an effective amount of an oxytocin peptide or a pharmaceutical composition comprising an effective amount of an oxytocin peptide. In one embodiment, the subject that has an impairment in executive function associated with a cognitive disorder selected from the group consisting of developmental disorder, aphasia, delirium, dementia, amnesia, executive dysfunction and cerebrovascular disease. In a further embodiment, the developmental disorder is selected from the group consisting of attention-deficit hyperactivity disorder, autism spectrum disorder, and Asperger's disorder. In yet another embodiment, the dementia is selected from the group consisting of Alzheimer's disease, cortical dementia, and subcortical dementia. In another embodiment, the subject has an impairment in executive function associated with a mental disorder selected from the group consisting of Tourette's syndrome, obsessive-compulsive disorder, unipolar and bipolar affective disorder, Schizotypal personality disorder, corpus callosum dysgenesis, impulsive personality disorder, acute stress disorder, traumatic brain injury and post-traumatic stress syndrome. In still another embodiment, the subject has an impairment in executive function associated with a disorder selected from the group consisting of Systemic Lupus Erythematosus, Parkinson's disease, rapid eye movement sleep behavior disorder, and Huntington's disease. In yet another embodiment, the subject is a person of at least 60 years in age, wherein the subject may or may not have an impairment in executive function, and wherein the subject does not have a cognitive or mental disorder. In a further embodiment, the subject is a person of at least 65 years in age and has an impairment in executive function. In yet another embodiment of any of the foregoing, the impairment of executive function is a mild impairment of executive function. In yet another embodiment of any of the foregoing the oxytocin peptide or the pharmaceutical composition comprising the oxytocin peptide is administered intranasally, intramuscularly, or intravenously. In a further embodiment, the oxytocin peptide or the pharmaceutical composition comprising the oxytocin peptide is administered intranasally. In still another embodiment of any of the foregoing the oxytocin peptide is administered at a dose of about 40-100 IU per day. In a further embodiment, the dose is at least two times per day at a dose of about 20-40 IU per administration. In still a further embodiment, the dose is about 40-80 IU per day. In yet another embodiment of any of the foregoing the oxytocin peptide is administered with at least one additional active agent, and wherein the at least one additional active agent is used to treat a mental or cognitive disorder. In a further embodiment, the at least one additional active agent is an antipsychotic agent, a stimulant, and/or antidementia agent. In yet another embodiment of any of the foregoing the oxytocin peptide is administered prior to or in conjunction with one or more programs to improve cognitive functions. In yet a further embodiment, the one or more programs to improve cognitive functions is selected from mindfulness meditation, multiple classification and ambiguous language activities, aerobic exercise, silent expression of conscious thoughts to oneself in coherent linguistic form, cognitive stimulation programs, working memory training, video games, and Multi-Attribute Task Battery tests.

The details of one or more embodiments of the disclosure are set forth in the accompanying drawings and the description below. Other features, objects, and advantages will be apparent from the description and drawings, and from the claims.

### DESCRIPTION OF DRAWINGS

FIG. 1 provides for the structure of oxytocin.

FIG. 2 provides a picture of a multi-hole operant box used to test animals for executive function.

FIG. 3 diagrams an operant setup with colored light cues used to test rat subjects for executive function.

FIG. 4 diagrams an operant paradigm used to test executive functioning. Two lights (e.g., red and green lights) are provided, where a first light (e.g., red light), the ‘target’, rewards a nose poke 80% of the time, while the second light (e.g., green light), the ‘nontarget’, rewards a nose poke only 20% of the time. After eight consecutive responses in which the ‘target’ stimulus is selected, the reward ratios of the two lights swap so that the ‘target’ switches from the first light to the second light, and the next block of testing begins.

FIG. 5A-B demonstrates the effects of a single administration of oxytocin on executive function. OT=oxytocin; BN=Brown Norway rat; LE=Long Evans rat. Vehicle and 0.04 mg/kg OT-treated BN rats exhibited fewer switches than vehicle-treated LE rats. OT (1 mg/kg) increased the total number of switches completed by BN rats vs. vehicle in the PRLT(A). Acute OT (1 mg/k/g) reduced the total trials to initial criterion vs. both vehicle-treated BN and LE rats) (B). As shown, Brown Norway rats—which exhibit poor executive function compared to LE rats, are improved after the administration of oxytocin. Moreover, the data demonstrate that the effect was dose dependent. In particular, Brown Norway rats who were treated with a larger dose (1.00 mg/kg) of Oxytocin exhibited better executive function than when lower doses (0.04 mg/kg and 0.20 mg/kg) were used. Data presented as mean±S.E.M. *=p<0.05 vs BN-1.00 mg/kg; #=p<0.05 vs LE-vehicle.

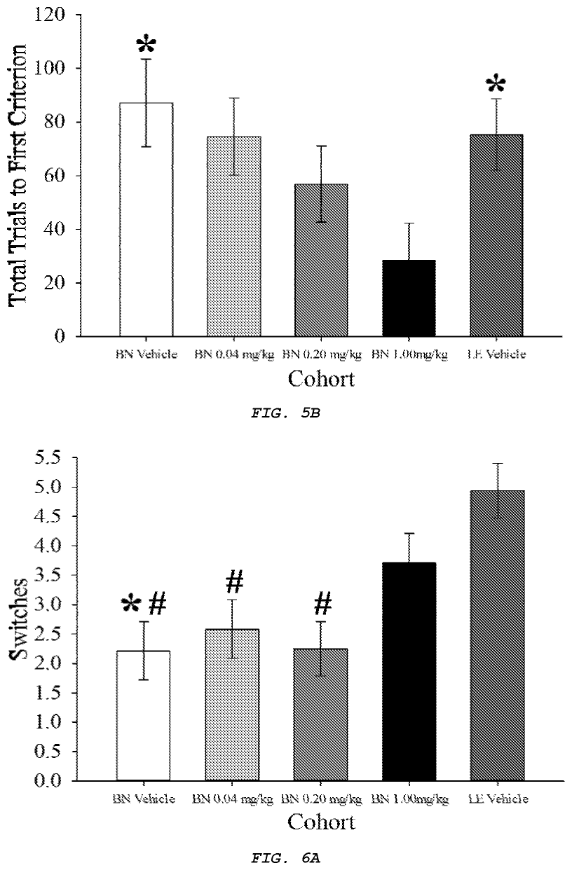

FIG. 6A-B demonstrates the effects of long-term administration of oxytocin on executive function. OT=oxytocin; BN=Brown Norway rat; LE=Long Evans rat. LE rats exhibited more switches than BN rats previously treated with vehicle, 0.04, or 0.2 mg/kg OT BN rats that were treated with 1 mg/kg OT exhibited significantly more switches than vehicle-treated BN rats (A). LE rats had few trials to first criterion than BN rats and 1 mg/kg OT appears to reduce trials to first criterion compared to vehicle in BN rats (B). As shown, Brown Norway rats that were treated with 1.0 mg/kg OT for 21 days exhibited improvement in executive function with persistent improvement when tested approximately one week or more after the discontinuation of OT administration indicating that oxytocin exerts a long-term effect on executive function. Data presented as mean±S.E.M. *=p<0.05 vs BN-1.00 mg/kg; #=p<0.05 vs LE-vehicle.

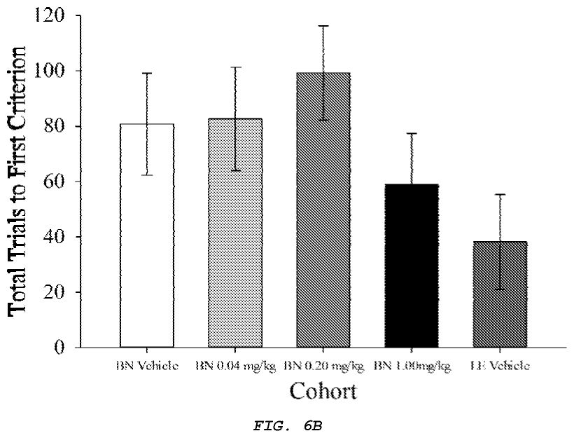

### DETAILED DESCRIPTION

As used herein and in the appended claims, the singular forms “a,” “an,” and “the” include plural referents unless the context clearly dictates otherwise. Thus, for example, reference to “a peptide” includes a plurality of such peptides and reference to “the cell” includes reference to one or more cells known to those skilled in the art, and so forth.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

Cognitive function is an intellectual process by which one becomes aware of, perceives, or comprehends ideas. It involves all aspects of perception, thinking, reasoning, and remembering.

There is ample evidence that alterations in brain structure and function are intimately tied to alterations in cognitive function and/or executive function. As such, many subjects with a mental disorder exhibit at least some form of impairment in cognition that is deterioration from a previous level of function. For certain mental disorders, cognitive dysfunction constitutes the core symptomatology. These cognitive disorders are ideally suited to being treated with the compounds disclosed herein. Examples of such cognitive disorders, include, but are not limited to, developmental disorders, including attention-deficit hyperactivity disorder (Shue et al., Brain and Cognition 20(1):104-124 (1992); Happe et al., Brain and Cognition 61(1):25-39 (2006); and Rubia et al., Human Brain Mapping 31(12):1823-1833 (2010)), autism spectrum disorders (Happe et al., Brain and Cognition 61(1):25-39 (2006)), and Asperger's disorder (Happe et al., Brain and Cognition 61(1):25-39 (2006)); aphasia (Gorno-Tempini et al., Annals of Neurology 55(3):335-346 (2004)); delirium (Girard et al., Crit Care Med 38(7):1513-1520 (2010)); dementia, including Alzheimer's disease (Albert et al., Alzheimer's & Dementia 7(3):270-279 (2011); Terry et al., Annals of Neurology 30(4):572-580 (1991); and Petersen et al., Arch Neurol. 56(3):303-308 (1999)), cortical dementia (Whitehouse et al., Science 215(4537):1237-1239 (1982)), and subcortical dementia (Cummings et al., Arch Neurol. 41(8):874-879 (1984)); amnesia (Janowsky et al., Behavioral Neuroscience 103(3):548-560 (1989)); executive dysfunction (Pereira et al., International Psychogeriatrics 20(6):1104-1115 (2008)); and cerebrovascular disease (Johnston et al., Ann Intern Med 140(4):237-247 (2004)). Additionally, the compounds disclosed herein can be used to treat subjects with other mental disorders that have been associated with some form of cognitive impairment, including, but not limited to, Tourette's syndrome (Watkins et al., Psychological Medicine 35(4):571-582 (2005)); obsessive-compulsive disorder (Head et al., Biological Psychiatry 25(7):929-937 (1989)); unipolar and bipolar affective disorder (Lars Vedel Kessing, Psychological Medicine 28(5):1027-1038 (1998)); depression disorders (Marazziti et al., European Journal of Pharmacology 626(1):83-86 (2010)); Schizotypal personality disorder (Siever et al., Schizophrenia Research 54:(1-2) (2002)); corpus callosum dysgenesis (Brown et al., Cognitive Neuropsychiatry 5(2):133-157 (2010)); impulsive personality disorder (Dolan et al., Psychological Medicine 32(1):105-117 (2002)); acute stress disorder (Amy F. T. Arnsten, Nature Reviews Neuroscience 10:410-422 (2009); and post-traumatic stress syndrome (Polak et al., Journal of Affective Disorders 141(1):11-21 (2012)). In a particular embodiment, a mental disorder as used herein comprises all of the mental disorders described herein except for schizophrenia.

Moreover, disorders not normally characterized as being a mental or psychological disorder, e.g., autoimmune and movement disorders, have also been shown to have some correlation with mild to significant cognitive impairment. For example, subjects with Systemic Lupus Erythematosus were found to have a high prevalence of cognitive impairment (see Carbotte et al., Journal of Nervous & Mental Disease 174(6):357-64 (1986); Maneeton et al., Asian Pac J. Allergy Immunol 28(1):77-83 (2010); and Meszaros et al., J. Clin Psychiatry 73(7):993-1001 (2012)), as well as subjects with Parkinson's disease (Litvan et al., Movement Disorders, 27(3):349-356 (2012)); rapid eye movement sleep behavior disorder (Gagnon et al., Annals of Neurology 66(1):39-47 (2009)); and Huntington's disease (Ho et al., Neurology 61(12):1702-1706 (2003)). Accordingly, disorders like the foregoing can be treated by administering the compounds disclosed herein.

Further, as people normally age, their brains change both biologically and psychologically. The basic cognitive functions most affected by age are attention and memory. Neither of these are unitary functions, however, and evidence suggests that some aspects of attention and memory hold up well with age while others show significant declines. Perception (although considered by many to be a precognitive function) also shows significant age-related declines attributable mainly to declining sensory capacities. Deficits at these early processing stages could affect cognitive functions later in the processing stream. Higher-level cognitive functions such as language processing and decision making may also be affected by age. These tasks naturally rely on more basic cognitive functions and will generally show deficits to the extent that those fundamental processes are impaired. Moreover, complex cognitive tasks may also depend on a set of executive functions, which manage and coordinate the various components of the tasks. Considerable evidence points to impairment of executive function as a key contributor to age-related declines in a range of cognitive tasks. Executive control is a multi-component construct that consists of a range of different processes that are involved in the planning, organization, coordination, implementation, and evaluation of many of our nonroutine activities. This so-called central executive plays a key role in virtually all aspects of cognition, allocating attentional resources among stimuli or tasks, inhibiting distracting or irrelevant information in working memory, formulating strategies for encoding and retrieval, and directing all manner of problem-solving, decision-making, and other goal-directed activities. Executive control is particularly important for novel tasks for which a set of habitual processes is not readily available. Executive function depends critically on prefrontal cortex, which exerts its broad-reaching controlling influence via extensive reciprocal connections with posterior cortical regions. Both structural and functional neuroimaging studies have revealed a preferential decline in older adults in volume and function of prefrontal brain regions. Accordingly, the compounds disclosed herein can be used to treat elderly patients that are exhibiting signs of cognitive impairment, and/or used prophylactically to prevent cognitive impairment in elderly patients.

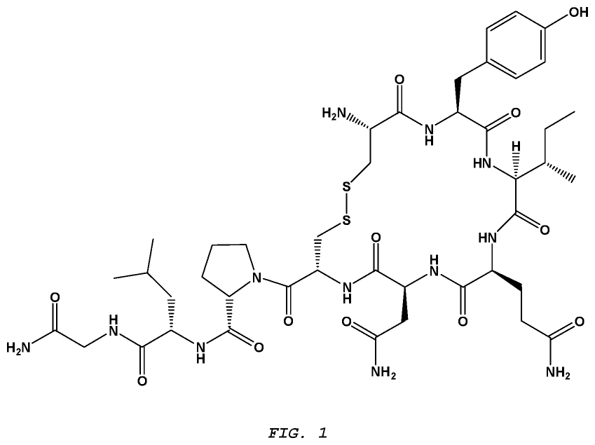

Oxytocin is a nine amino acid cyclic peptide hormone with two cysteine residues that form a disulfide bridge between positions 1 and 6 (see, FIG. 1). Human oxytocin comprises the sequence Cys-Tyr-Ile-Gln-Asn-Cys-Pro-Leu-Gly (SEQ ID NO:1). Oxytocin is released from the pituitary gland and stimulates the contraction of smooth muscle of the uterus during labor and facilitates release of milk from the breast during nursing. Oxytocin has historically been used to induce labor.

As provided herein, the disclosure provides for compounds that have biological activity that is similar to or identical to natural oxytocin. In a particular embodiment, a compound of the disclosure or a compound disclosed herein refers to oxytocin or a “oxytocin peptide.”

As used herein, “oxytocin” or “oxytocin peptide” refers to a substance having biological activity associated with natural oxytocin. Oxytocin or oxytocin peptide can be a naturally occurring endogenous peptide, fragments, analogues or derivatives thereof. Oxytocin or oxytocin peptide can also be a non-endogenous peptide, fragments, analogues or derivatives thereof. An oxytocin peptide includes both natural or synthetic, therapeutically or prophylactically active, peptide fragments, peptide analogues, and chemically modified derivatives or salts of active peptides. There are processes described for the production of oxytocin, see for example U.S. Pat. Nos. 2,938,891 and 3,076,797. In addition, oxytocin is commercially available. A variety of peptide analogues and derivatives are available and others can be contemplated for use within the disclosure and can be produced and tested for biological activity according to known methods. Oxytocin analogues may included, but are not limited to, 4-threonine-1-hydroxy-deaminooxytocin, 4-serine, 8-isoleucine-oxytocin, 9-deamidooxytocin, 7-D-proline-oxytocin and its deamino analog, (2,4-diisoleucine)-oxytocin, deamino oxytocin analog, 1-deamino-1-monocarba-E12-Tyr(OMe)]-OT(dCOMOT), carbetocin, 4-threonine, 7-glycine-oxytocin (TG-OT), oxypressin, deamino-6-carba-oxytoxin (dC60), deamino-1 monocarba-(2-O-methyltyrosine)-oxytocin [d(COMOT)]); [Thr4-Gly7]-oxytocin (TG-OT); oxypressin; Ile-conopressin; atosiban; deamino-6-carba-oxytoxin (dC60), d[Lys(8) (5/6C-Fluorescein)]VT, d[Thr(4), Lys(8) (5/6C-Fluorescein)]VT, [HO(I)][Lys(8) (5/6C-Fluorescein)]VT, [HO(I)][Thr(4), Lys(8) (5/6CFluorescein)]VT, d[Om(8) (5/6C-Fluorescein)]VT, d[Thr(4), Om(8) (5/6C-Fluorescein)]VT, [HO(1)][Om(8)(5/6C-Fluorescein)]VT, [HO(I)][Thr(4), Om(8) (5/6C-Fluorescein)]VT, desmopressin, and 1-deamino-oxytocin in which the disulfide bridge between residues 1 and 6 is replaced by a thioether, L-371,257 and the related series of compounds containing an ortho-trigluoroethoxyphenylacetyl core such as L-374,943. Oxytocin peptide and polypeptide useful in the methods and compositions of the disclosure include peptides that are obtainable by partial substitution, addition, or deletion of amino acids within a naturally occurring or native peptide sequence.

As used herein, “oxytocin analogues and derivatives” refers to any peptide analogous of naturally occurring oxytocin wherein one or more amino acids within the peptide have been substituted, deleted, or inserted. The term also refers to any peptide wherein one or more amino acids have been modified, for example by chemical modification. In general, the term covers all peptides which exhibit oxytocin activity but which may, if desired, have a different potency or pharmacological profile. Peptides can be chemically modified, for example, by amidation of the carboxyl terminus (—NH₂), the use of D amino acids in the peptide, incorporation of small non-peptidyl moieties, as well as the modification of the amino acids themselves (e.g., alkylation or esterification of side chain R-groups). Such analogues, derivatives and fragments should substantially retain the desired biological activity of the native oxytocin peptide.

In still other embodiments the oxytocin analogs are fragments of oxytocin, for example, peptide cleavage products. Such fragments may be chemically synthesized or derived by any known means. Oxytocin fragments of the disclosure retain bioactivity similar to or greater than wild-type oxytocin. Such fragments may be capable of crossing the blood brain barrier.

In another embodiment of the disclosure, oxytocin analogs or mimetics are synthetic molecules that retain oxytocin bioactivity. Such analog or mimetic molecules are capable of acting in a manner similar to endogenous oxytocin, including binding the oxytocin receptor. Analogs of this type may be derivatives of oxytocin or have completely new molecular structures.

In another embodiment oxytocin analogs can be modified for increased stability, enhancement of transport across the blood brain barrier, retention in the brain once they have crossed the blood brain barrier or a combination of the foregoing. Modifications to increase stability and enhance blood brain barrier transport may include, but are not limited to, esterification with steroids, such as cholesteryl, or esterification with fatty alcohols, such as C-8 to C-22 alcohols. Modifications to increase retention in the brain include, but are not limited to, covalent attachment of 1,4-dihydrotrigonellinate and other redox sensitive functionalities, such as quinones and derivatives such as benzoquinones, naphthoquinones, indolequinones, nitroheterocycles such as nitrobenzyl, nitrofurans, and nitroimadzole derivatives.

The peptides/polypeptides described and/or contemplated herein can be prepared by chemical synthesis using either automated or manual solid phase synthetic technologies, generally known in the art. The peptides can also be prepared using molecular recombinant techniques known in the art.

A polypeptide or peptide of the disclosure may be prepared by culturing transformed host cells under culture conditions suitable to express the recombinant polypeptide. The resulting expressed polypeptide may then be purified from such culture (e.g., from culture medium or cell extracts) using known purification processes, such as gel filtration and ion exchange chromatography. The purification of a polypeptide may also include an affinity column containing agents which will bind to the polypeptide; one or more column steps over such affinity resins as concanavalin A-agarose, Heparin-Toyopearl or Cibacrom blue 3GA Sepharose; one or more steps involving hydrophobic interaction chromatography using such resins as phenyl ether, butyl ether, or propyl ether; or immunoaffinity chromatography. Alternatively, a polypeptide of the disclosure may also be expressed in a form that will facilitate purification. For example, it may be expressed as a fusion polypeptide, such as those of maltose binding polypeptide (MBP), glutathione-S-transferase (GST), thioredoxin (TRX) or polyhistidine. Kits for expression and purification of such fusion polypeptides are commercially available from New England BioLab (Beverly, Mass.), Pharmacia (Piscataway, N.J.) and InVitrogen, respectively. A polypeptide can also be tagged with an epitope and subsequently purified by using a specific antibody directed to such epitope. One such epitope (“Flag”) is commercially available. Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous isolated recombinant polypeptide. A polypeptide thus purified is substantially free of other mammalian polypeptides and is defined in accordance with the disclosure as a “substantially purified polypeptide. A polypeptide of the disclosure may also be expressed as a product of transgenic animals, e.g., as a component of the milk of transgenic cows, goats, pigs, or sheep which are characterized by somatic or germ cells containing a polynucleotide encoding the polypeptide.

It is also possible to utilize an affinity column comprising a monoclonal antibody generated against an oxytocin peptide of the disclosure, to affinity-purify an expressed polypeptide. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the disclosure. In this embodiment of the disclosure, an anti-polypeptide antibody of the disclosure or other polypeptides that can interact with a polypeptide of the disclosure, can be bound to a solid phase support such as a column chromatography matrix or a similar substrate suitable for identifying, separating, or purifying cells that express polypeptides of the disclosure on their surface.

A polypeptide may also be produced by known conventional chemical synthesis. Methods for constructing polypeptides of the disclosure by synthetic means are known to those skilled in the art. The synthetically constructed polypeptides, by virtue of sharing primary, secondary or tertiary structural and/or conformational characteristics with native polypeptides may possess biological properties in common therewith, including polypeptide activity. Thus, they may be employed as biologically active or immunological substitutes for natural, purified polypeptides in screening of therapeutic compounds and in immunological processes for the development of antibodies.

The desired degree of purity depends on the intended use of a polypeptide. A relatively high degree of purity is desired when a polypeptide is to be administered in vivo, for example. In such a case, polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. A polypeptide of the disclosure is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

“Executive function” refers to high level cognitive functions that involves the ability to mentally represent information or a problem, plan a solution by selecting a strategy, maintain the strategy in short term memory in order to perform it and monitor the results in order to modify the strategy. Probabilistic learning is one measurable feature of executive functioning, and refers to a gradual feedback-based learning of probabilistically related cue-outcome associations, without the necessity of conscious appreciation of the rules or strategies. Put simply it is the ability to optimize behavior in situations where there are probabilistic chances of certain outcomes to each behavior and the probabilities change over time. For example, if a person presented with two different colored buttons to press (red and blue) to avoid receiving an electric shock, the person may quickly learn to press the red colored button each time if it prevents the shock 60% of the time it is pressed, whereas the blue one only prevents it 40% of the time. However, if the probabilities become reversed, intact probabilistic learning would make it possible for the person to recognize the new probability and modify his behavior by switching his strategy to press the blue button each time. Probabilistic learning is a certain type of reinforcement learning. Reinforcement learning is generally defined as the modification of behavior based on past experience of the positive and/or negative consequences of particular predictive events (stimuli or actions). Probabilistic learning has been associated with, among other areas, the prefrontal cortex of the brain, the area that is most associated with executive function. Several neuropsychiatric disorders such as schizophrenia and autism are associated with deficient probabilistic learning.

Executive function can be considered the ability to switch one's thinking (cognition) (or train of thought) as an adaptation to the demands of stimuli. In neuroscience, the term is sometimes referred to as “attention switching,” “cognitive shifting,” “mental flexibility,” “set shifting,” and “task switching.” Executive function is useful in that it helps people adapt to novel scenarios and information such as: moving to a foreign country, unexpected demands in the workplace, and/or a last-minute change of plans.

Executive function is immensely important for shifting attention and thoughts quickly. Those with low levels of executive function are unable to shift from one concept to another, and often become “stuck” in a single train of thought or aspect of focus (i.e., centration). In addition to improving a person's ability to adapt when faced with novel stimuli, executive function is associated with improved brain functioning. It allows the brain to function more efficiently, with various regions operating in orchestra. Those with high levels of executive function tend to have superior comprehension and fluency associated with reading, higher levels of fluid intelligence, and an expanded sense of awareness.

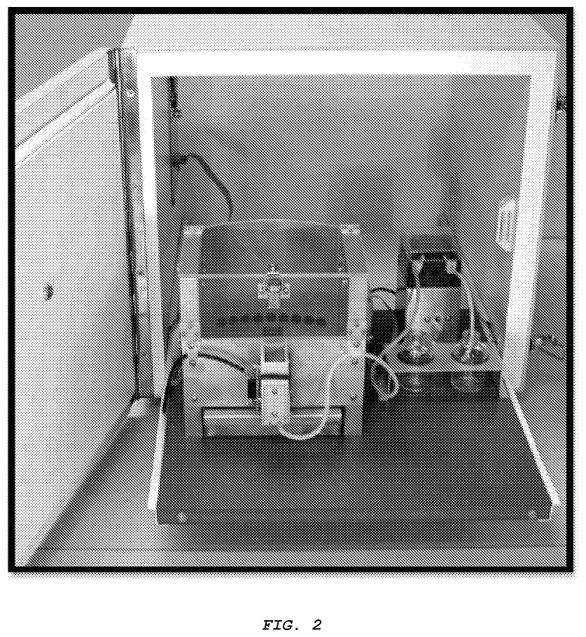

Probabilistic learning, an aspect of executive function, can be assessed in rodents using a probabilistic reversal learning task (PRLT). A PRLT is typically a 1-hour test using two recessed lights in the back of the operant chamber as stimuli (see, FIGS. 2, 3 and 4). After illumination and subsequent extinguishing of the magazine light via nose-poke, a 2 second inter-trial interval occurs, which is then immediately followed by a 10 second period during which two apertures are illuminated wherein the rat can respond via nose-poke. One of the two stimuli is designated the target stimulus, and responses to it are rewarded more frequently (e.g., rewarded 80% of the time) (aperture light extinguished and reward delivered into the magazine) and punished less frequently (e.g., 20% of the time) (aperture light extinguished and house light illuminated for a 4-second timeout period during which no reward was delivered). The other stimulus was designated the non-target stimulus, to which responses are rewarded less frequently (e.g., 20% of the time) and punished more frequently (80% of the time). Initial target and non-target locations are alternated among the testing chambers such that the first target of the session is, for example, the left light in four of the eight chambers, and the right in the others. Criterion for demonstrating acquisition is typically designated as 8 consecutive responses to the target stimulus. After first criterion acquisition, the target and non-target locations are reversed. The subject then has to recognize that this change, or “switch,” had occurred and adjust its responses accordingly. After another 8 consecutive correct responses, the ratios are switched again. This pattern is repeated for the remainder of the 1-hour session. The number of switches the subject is able to complete within the testing period was the primary outcome variable of PRLT to measure reversal learning. An additional primary outcome measure, trials to first criterion, provided a measure of initial reinforcement learning without the confound of reversal learning included.

Secondary outcome variables included omissions (no responses to stimuli within 10 seconds of illumination) and premature responses (response to stimuli apertures during the ITI), both of which resulted in a 4 second punishing time out. Several latency measures included mean target latency (millisecond (ms) to respond in the target stimulus), mean non-target latency (ms to respond in the non-target stimulus), and mean reward latency (ms to collect the reward).

Using the devices described herein and those known in the art, oxytocin and peptide analogs or mimetics can be tested on animal models to determine their biological effect. In particular, the peptide or mimetic can be administered and then the animal texted for executive function as described herein. Moreover, dosing can be assessed in a similar manner. Finally, disease models of cognitive functions and mental disorders can be assessed using the systems described herein, both before and after administration of an oxytocin peptide, analog or mimetics, wherein an improvement in probabilistic learning is indicative or an agent treating a disease or disorder and/or improving executive function.

The disclosure provides a method of treating a subject with cognitive impairment or prophylactically preventing the occurrence of cognitive impairment in a subject comprising administering one or more compounds disclosed herein to the subject. In a particular embodiment, the one or more compounds are selected from a naturally occurring purified form of oxytocin, a recombinant form of oxytocin and analogs or mimetics of oxytocin, a chemically modified form of oxytocin or a combination thereof. In a further embodiment, a subject to be treated by a compound disclosed herein can have a mental disorder. In a further embodiment, a subject to be treated by a compound disclosed herein has a cognitive mental disorder. In yet a further embodiment, a subject to be treated by a compound disclosed herein has cognitive impairment as a result of aging, wherein the subject is at least 50 years old, at least 55 years old, at least 60 years old, at least 65 years old, at least 70 years old, at least 75 years old or at least 80 years old. In another embodiment, the subject to be treated has a deficiency of executive function either due to environmental factors, genetics, disease or age. In an alternate embodiment, a compound disclosed herein is used to prophylactically prevent the occurrence of cognitive impairment in an elderly subject by administering one or more compounds disclosed herein, wherein the elderly subject is at least 45 years old, at least 50 years old, at least 55 years old, at least 60 years old, at least 65 years old, at least 70 years, at least 75 years old, or at least 80 years old. In another embodiment, the compound disclosed herein is administered parenterally, intramuscularly, or intranasally. In a particular embodiment, one or more compounds disclosed herein are administered intranasally. Generally, intranasal delivery improves uptake, and patient compliance. In one embodiment, a method disclosed herein comprises delivering one or more compounds of the disclosure intranasally at least twice per day. In another embodiment, a compound disclosed herein is delivered at least twice per day at a dose of about 20-50 IU (international units) per administration (e.g., about 40-100 IU per day). Doses may range from about 10-80 IU per administration and will depend upon various factors readily identifiable to a physician (e.g., body weight, route of administration, formulation, severity of a disease or disorder and the like). Accordingly, the total dose for a subject may be about 20-160 (e.g., 20, 30, 40, 50, 60, 70, 80, 90, 100, 110, 120, 130, 140, 150 or 160) IU of oxytocin per day. In another embodiment, a compound of the disclosure is administered intranasally at a dose of about 40-100 IU per day. The dosing may be one or more times per day. The dosing may continue for several days, weeks, months or years. In another embodiment, one or more compounds disclosed herein are administered as an adjunctive therapy to standard/current therapy for a mental disorder. In yet another embodiment, one or more compounds disclosed herein are administered chronically or long term for at least 3 weeks or longer at least twice a day as described above. In yet other embodiments, one or more compounds disclosed herein may be administered intraperitoneally, intravascularly, intramuscularly, orally, and the like, in either delayed or sustained release formulations or immediate release formulations.

The disclosure also provides a method of improving executive function of a subject by administering one or more compounds disclosed herein. In a particular embodiment, the subject has impaired executive function. In an alternate embodiment, the subject does not have impaired executive function. In a further embodiment, one or more compounds are administered to prophylactically prevent the impairment of executive function in a subject, such as an elderly subject. In a particular embodiment, the subject is a subject presenting with characteristics of a mental disorder, or having mental disorder. In a further embodiment, the subject is a subject presenting with characteristics of a cognitive disorder, or having a cognitive disorder. In one embodiment, a compound disclosed herein is administered intranasally, mucosally, sublingually and the like to a subject in order to improve executive function. In yet a further embodiment, a method to improve executive function of a subject comprises administering one or more compounds disclosed herein to a subject intranasally at least twice per day. In another embodiment, a method to improve executive function of a subject comprises administering a compound disclosed herein at least twice per day at a dose of about 20-40 IU (international units) per administration (e.g., about 40-100 IU per day). In yet another embodiment, a method to improve executive function of a subject comprises administering a compound disclosed herein intranasally at a dose of about 40-100 IU per day. The dosing may be one or more times per day. The dosing may continue for several days, weeks, months or years. In yet another embodiment, a compound disclosed herein is administered chronically or long term for at least 3 weeks or longer at least twice a day as described above. In yet other embodiments, one or more compounds disclosed herein may be administered intraperitoneally, intravascularly, intramuscularly, orally, and the like, in either delayed or sustained release formulations or immediate release formulations.

Impairments in cognitive performance and memory have been reported in humans treated with oxytocin, accordingly, the data presented herein is unexpected. The disclosure demonstrates that when oxytocin was administered to animal subjects, the executive function of the animal subjects significantly improved. Thus, the methods of the disclosure comprise treating a mammal including a human with an effective amount of compound disclosed herein so as to improve the cognitive function (e.g., executive function) of the mammal. In particular, to mammals that have impaired cognitive function as a result of an underlying mental disorder or due to effects of natural aging.

The effect or an effective dose of a compound disclosed herein can be measured by using various recognized testing methods. For example, tests such as A-Not-B Task, Multiple Classification Card Sorting Task, Wisconsin Card Sorting Test, Stroop Test, Symbol Digit Modalities Test Screening test, Ruff Figural Fluency Test, Repeatable Battery for the Assessment of Neuropsychological Status, Paced Auditory Serial Attention Test, Kaufman Short Neuropsychological Assessment, Halstead Category Test, Dementia Rating Scale, Delis-Kaplan Executive Function System, and Cognitive Symptom Checklists, can be used.

The compounds of the disclosure can be formulated for deliver by admixture with pharmaceutically acceptable non-toxic excipients or carriers. Mention may be made, as examples of pharmaceutically acceptable salts, of the addition salts with inorganic or organic acids (such as acetate, trifluoroacetate, propionate, succinate, benzoate, fumarate, maleate, oxalate, methanesulphonate, isethionate, theophyllinacetate, salicylate, methylenebis-β-oxynaphthoate, hydrochloride, sulphate, nitrate and phosphate), the salts with alkali metals (sodium, potassium or lithium) or with alkaline-earth metals (calcium or magnesium), the ammonium salt or the salts of nitrogenous bases (ethanolamine, trimethylamine, methylamine, piperidine, benzylamine, N-benzyl-α-phenethylamine, choline, arginine, leucine, lysine or N-methylglucamine).

The disclosure provides pharmaceutical compositions of oxytocin peptides or their salts. The oxytocin peptide or their physiologically acceptable salts or solvates, may be formulated for administration for injection, or for oral, topical, nasal, inhalation, insufflation (either through the mouth or the nose) buccal, parenteral, rectal administration or other forms of administration. The disclosure provides pharmaceutical compositions comprising effective amounts of an oxytocin peptide together with pharmaceutically acceptable diluents, preservatives, solubilizers, emulsifiers, adjuvants, excipients and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., thimerosal, benzyl alcohol) and bulking substances (e.g., lactose, mannitol).

The compositions may also be incorporated into particulate preparations of polymeric compounds such as polylactic acid, polyglycolic acid, and the like or liposomes. Hyaluronic acid may also be used. Biocompatible absorbable polymers may be selected from the group consisting of aliphatic polyesters, copolymers and blends, which include, but are not limited to, homopolymers and copolymers of lactide (which include D-, L-, lactic acid and D-, L- and meso lactide), glycolide (including glycolic acid), epsilon-caprolactone, p-dioxanone (1,4-dioxan-2-one), alkyl substituted derivatives of p-dioxanone (i.e., 6,6-dimethyl-1,4-dioxan-2-one), triethylene carbonate (1,3-dioxan-2-one), alkyl substituted derivatives of 1,3-dioxanone, delta-valerolactone, beta-butyrolactone, gamma-butyrolactone, epsilon-decala tone, hydroxybutyrate, hydroxyvalerate, 1,4-dioxepan-2-one and its dimer 1,5,8,12-tetraoxacyclotetradecane-7,14 dione, 1,5-dioxepan-2-one, and polymer blends thereof.

Such compositions may influence physical state, stability, rate of in vivo release, and rate of in vivo clearance. See, e.g., Remington s Pharmaceutical Sciences, 18th ed., (1990, Mack Publishing Co., Easton, Pa. 18042) pages 1435-1712). The compositions may be prepared in liquid form, or be in dried powder, such as lyophilized form.

Contemplated for use herein are oral solid dosage forms, which are disclosed generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets or pellets. Also, liposomal or proteinoid encapsulation may be used to formulate compositions. Liposomal encapsulation may be used and the liposomes may be derivatized with various polymers. A description of possible solid dosage forms for the therapeutic is given by Marshall, K. In: Modern Pharmaceutics Edited by G. S. Banker and C. T. Rhodes Chapter 10, 1979). In general, the formulation will include an Oxytocin peptide and inert ingredients (which allow for protection against the stomach environment and release of the biologically active material in the intestine).

To ensure full gastric resistance a coating impermeable to at least pH 5.0 is useful. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L3OD, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings that make the tablet easier to swallow. Capsules may consist of a hard shell (such as gelatin) for delivery of dry therapeutic, i.e., powder; for liquid forms, a soft gelatin shell may be used. The shell material of cachets may be thick starch or other edible paper. For pills, lozenges, molded tablets or tablet triturates, moist massing techniques can be used.

The therapeutic can be included in the formulation as fine multi-particulates in the form of granules or pellets. The formulation of the material for capsule administration can also be as a powder, lightly compressed plugs or even as tablets. The therapeutic can also be prepared by compression.

Colorants and flavoring agents may all be included. For example, the peptide (or derivative) may be formulated (such as by liposome or microsphere encapsulation) and then further contained within an edible product, such as a refrigerated beverage containing colorants and flavoring agents.

One may dilute or increase the volume of the therapeutic with an inert material or filler. These diluents or fillers can include carbohydrates, especially mannitol, anhydrous lactose, cellulose (e.g., microcrystalline cellulose), sucrose, calcium hydrogen phosphate modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include, but are not limited to, starch (e.g., potato starch or the commercial disintegrant based on starch, Explotab). Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch (e.g., pregelatinised maize starch) and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to granulate the therapeutic.

An anti-frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes, talc and silica. Soluble lubricants may also be used such as sodium lauryl sulfate, magnesium lauryl sulfate, polyethylene glycol of various molecular weights, Carbowax 4000 and 6000.

Glidants that can improve the flow properties of the drug during formulation and to aid rearrangement during compression can be added. The glidants can include starch, talc, pyrogenic silica and hydrated silicoaluminate.

To aid dissolution of the therapeutic into the aqueous environment a surfactant can be added as a wetting agent. Surfactants may include anionic detergents such as sodium lauryl sulfate, dioctyl sodium sulfosuccinate and dioctyl sodium sulfonate. Cationic detergents can be used and can include benzalkonium chloride or benzethonium chloride. The list of potential non-ionic detergents that can be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants can be present n the formulation of the protein or derivative either alone or as a mixture in different ratios.

Additives that potentially enhance uptake of the agent are, for example, the fatty acids oleic acid, linoleic acid and linolenic acid.

Controlled release oral formulation may be desirable. The agent can be incorporated into an inert matrix that permits release by either diffusion or leaching mechanisms, e.g., gums. Slowly degenerating matrices may also be incorporated into the formulation. Some enteric coatings also have a delayed release effect.

Other coatings may be used for the formulation. These include a variety of sugars that can be applied in a coating pan. The therapeutic agent can also be given in a film coated tablet and the materials used in this instance are divided into two groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials can be used to provide the optimum film coating. Film coating may be carried out in a pan-coater or in a fluidized bed or by compression coating.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The compounds disclosed herein may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations disclosed previously, the compounds disclosed herein may also be formulated as a depot preparation. Such long acting formulations may be administered by implantation (for example subcutaneously or intramuscularly) or by intramuscular injection. Thus, for example, the compounds disclosed herein may be formulated with suitable polymeric or hydrophobic materials (for example as an emulsion in an acceptable oil) or ion exchange resins, or as sparingly soluble derivatives, for example, as a soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

Toxicity and therapeutic efficacy of the compounds disclosed herein can be determined by standard pharmaceutical procedures in cell cultures or experimental animal/animal models (such as those described herein), e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED₅₀ with little or no toxicity. The dosage may vary within this range depending upon the dosage form employed and the route of administration utilized. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

A compound disclosed herein and components of a therapeutic composition may be introduced parenterally, topically, or transmucosally, e.g., orally, nasally, or rectally, or transdermally. Parenteral administration includes, for example, intravenous injection, intra-arteriole, intramuscular, intradermal, subcutaneous, intraperitoneal, intraventricular, and intracranial administration.

Because many of compounds of the disclosure are capable of crossing the blood brain barrier, the compounds are suitable for oral, parenteral or intravenous administration. Alternatively, the compound can be modified or otherwise altered so that it can cross or be transported across the blood brain barrier. Many strategies known in the art are available for molecules crossing the blood-brain barrier, including but not limited to, increasing the hydrophobic nature of a molecule; introducing the molecule as a conjugate to a carrier, such as transferring, targeted to a receptor in the blood-brain barrier, or to docosahexaenoic acid and the like.

In another embodiment, a compound disclosed herein may be administered by surgical intervention including a procedure of drilling a small hole in the skull to administer the agent.

In another embodiment, a compound of the disclosure can be administered intracranially or intraventricularly. In another embodiment, osmotic disruption of the blood-brain barrier can be used to effect delivery of the compound to the brain (Nilayer et al., Proc. Natl. Acad. Sci. USA 92:9829-9833 (1995)). In yet another embodiment, a compound of the disclosure can be administered in a liposome targeted to the blood-brain barrier. Administration of pharmaceutical agents in liposomes are known (see Langer, Science 249:1527-1533 (1990); Treat et al., Liposomes in the Therapy of infectious Disease and Cancer, Lopez-Berestein and Fidler (eds.), Liss, New York, pp. pp. 317-327 and 353-365 (1989).

Some predictions have been made concerning the ability of molecules to pass through the blood-brain barrier, the rate and extent of entry of a compound of the disclosure or a formulation comprising a compound disclosed herein into the brain are generally considered to be determined by partition coefficient, ionization constant(s), and molecular size.

In another embodiment, a therapeutic formulation comprising an oxytocin peptide can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 317-327 and 353-365 (1989)).

In another embodiment, a therapeutic formulation comprising a compound of the disclosure can be delivered in a controlled release system. For example, the oxytocin peptide may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

In addition, any of the materials described herein can be administered to any part of the mammal's body including, without limitation, brain, spinal fluid, blood stream, lungs, nasal cavity, intestines, stomach, muscle tissues, skin, peritoneal cavity, and the like. Thus, a compound (e.g., an oxytocin peptide) can be administered by intravenous, intraperitoneal, intramuscular, subcutaneous, extracranial, intrathecal, and intradermal injection, by oral administration, by inhalation, or by gradual perfusion over time. For example, an aerosol preparation can be given to a mammal by inhalation. It is noted that the duration of treatment with the materials described herein can be any length of time from as short as one day to as long as a lifetime (e.g., many years). For example, a formulation comprising a compound of the disclosure can be administered once (or twice, three times, etc.) daily, weekly, monthly, or yearly.

Preparations for administration can include sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water as well as alcohol, saline, and buffered solutions. Preservatives, flavorings, and other additives such as, for example, antimicrobials, anti-oxidants, chelating agents, inert gases, and the like may also be present.

A compound of the disclosure or a pharmaceutical composition comprising a compound of the disclosure may be dispensed intranasally or mucosally as a powdered or liquid nasal spray, suspension, nose drops, a gel, film or ointment, through a tube or catheter, by syringe, by packtail, by pledget (a small flat absorbent pad), by nasal tampon or by submucosal infusion. Nasal drug delivery can be carried out using devices including, but not limited to, unit dose containers, pump sprays, droppers, squeeze bottles, airless and preservative-free sprays, nebulizers (devices used to change liquid medication to an aerosol particulate form), metered dose inhalers, and pressurized metered dose inhalers. It is important that the delivery device protect a compound from contamination and chemical degradation. The device should also avoid leaching or absorption as well as provide an appropriate environment for storage. Each agent needs to be evaluated to determine which nasal drug delivery system is most appropriate. Nasal drug delivery systems are known in the art and several are commercially available.

A compound of the disclosure or a pharmaceutical composition comprising a compound disclosed herein may be conveniently delivered in the form of an aerosol spray using a pressurized pack or a nebulizer and a suitable propellant including, but not limited to, dichlorodifluoromethane, trichlorofluoromethane, dichlorotetrafluoroethane, hydrocarbons, compressed air, nitrogen or carbon dioxide. An aerosol system requires the propellant to be inert towards the pharmaceutical composition. In the case of a pressurized aerosol, the dosage unit may be controlled by providing a valve to deliver an accurately metered amount.

The means to deliver a compound of the disclosure or pharmaceutical composition comprising a compound disclosed herein to the nasal cavity as a powder can be in a form such as microspheres delivered by a nasal insufflator device (a device to blow a gas, powder, or vapor into a cavity of the body) or pressurized aerosol canister. The insufflator produces a finely divided cloud of the dry powder or microspheres. The insufflator may be provided with means to ensure administration of a substantially metered amount of the pharmaceutical composition. The powder or microspheres should be administered in a dry, air-dispensable form. The powder or microspheres may be used directly with an insufflator which is provided with a bottle or container for the powder or microspheres. Alternatively, the powder or microspheres may be filled into a capsule such as a gelatin capsule, or other single dose device adapted for nasal administration. The insufflator can have means such as a needle to break open the capsule or other device to provide holes through which jets of the powdery composition can be delivered to the nasal cavity.

Nasal delivery devices can be constructed or modified to dispense a compound of the disclosure or a pharmaceutical composition comprising a compound disclosed herein wherein the compound or the composition is delivered predominantly to the inferior two-thirds of the nasal cavity. For example, the angle of dispersion from a delivery device such as a nebulizer or an insufflator can be set so that the pharmaceutical composition is mechanically directed to the inferior two-thirds of the nasal cavity, and preferably away from the superior region of the nasal cavity. Alternatively, a compound of the disclosure or a pharmaceutical composition comprising a compound disclosed herein can be delivered to the inferior two-thirds of the nasal cavity by direct placement of the composition in the nasal cavity, for example, with a gel, an ointment, a nasal tampon, a dropper, or a bioadhesive strip.

Thus, in some embodiments of the disclosure, the methods comprise administering to an individual a compound of the disclosure or pharmaceutical composition comprising a compound disclosed herein wherein administration to the nasal cavity is by a nasal delivery device. The nasal delivery device can include, but is not limited to, unit dose containers, pump sprays, droppers, squeeze bottles, airless and preservative-free sprays, nebulizers, dose inhalers, pressurized dose inhalers, insufflators, and bi-directional devices. The nasal delivery device can be metered to administer an accurate effective dosage amount to the nasal cavity. The nasal delivery device can be for single unit delivery or multiple unit delivery. In some embodiments of the disclosure, the nasal delivery device can be constructed whereby the angle of dispersion of a pharmaceutical composition is mechanically directed towards the inferior two-thirds of the nasal cavity thereby minimizing delivery to the olfactory region. In some embodiments of the disclosure, the nasal delivery device may be activated only on exhalation, thus limiting the inhalation induced and potentially undesirable distribution of the pharmaceutical composition. In some embodiments of the disclosure, the pharmaceutical composition is a gel, film, cream, ointment, impregnated in a nasal tampon or bioadhesive strip whereby the composition is placed in the inferior two-thirds of the nasal cavity. In some embodiments of the disclosure, the methods include intranasal administration of a compound of the disclosure or a pharmaceutical composition comprising a compound disclosed herein wherein the administration uses a nasal delivery device with an angle of dispersion that mechanically directs the agent to the inferior two-thirds of the nasal cavity wherein the compound of the disclosure is administered after a vasoconstrictor. In some embodiments of the disclosure, the methods include intranasal administration of a compound of the disclosure or pharmaceutical composition comprising a compound disclosed herein wherein the administration uses a nasal delivery device with an angle of dispersion that mechanically directs the agent to the inferior two-thirds of the nasal cavity wherein the compound is co-administered with a vasoconstrictor.

As used herein, “mucosal administration” or “administered transmucosally” refers to delivery to the mucosal surfaces of the nose, nasal passageways, nasal cavity; the mucosal surfaces of the oral cavity including the gingiva (gums), the floor of the oral cavity, the cheeks, the lips, the tongue, the teeth; and the mucosal surfaces of or around the eye including the conjunctiva, the lacrimal gland, the nasolacrimal ducts, the mucosa of the upper or lower eyelid and the eye.

Intranasal drug delivery has been a topic of research and development for many years, although it has been only within the past decade that carrier systems have been devised which make delivery of substances effective. (Sayani and Chien (1996) Critical Reviews in Therapeutic Drug Carrier Systems, 13:85-184). Intranasal delivery has a number of advantageous features including comparatively high bioavailability, rapid kinetics of absorption and avoidance of a first-pass effect in the liver. In regard to patient compliance and ease of use, intranasal administration provides a simple, rapid and non-invasive mode of application. In some embodiments, intranasal administration can allow for delivery of a compound of the disclosure to the nasal cavity and in other embodiments, intranasal administration can allow for targeted delivery to the trigeminal nerve. Targeted delivery to the trigeminal nerve and preferably not the olfactory region can reduce the amount of drug entering the CNS or systemic circulation thereby reducing or eliminating potential undesirable CNS effects or systemic side effects. Targeted delivery to the trigeminal nerve can also reduce the effective dosage necessary to achieve analgesia in the facial or head regions wherein lower effective dosages will further reduce any potential CNS or systemic side effects.

As used herein, “intranasal administration” or “administered intranasally” refers to delivery to the nose, nasal passageways or nasal cavity by spray, drops, powder, gel, film, inhalant or other means.

The nasal cavity contains turbinate bones which protrude into the nasal cavity and generally separate it into three regions. As used herein, the “inferior region of the nasal cavity” refers to the portion of the nasal cavity where the middle and inferior turbinate bones protrude and is a region of the nasal cavity that is innervated by the trigeminal nerve system. The superior area of the nasal cavity is defined by the superior turbinate bone wherein the olfactory region is located.

A compound of the disclosure is administered in a dose sufficient to provide a therapeutically effective amount to an individual to improve the cognitive function, e.g., executive function, of a subject. A therapeutically effective dose of a compound of the disclosure can be determined empirically and depends on the type of treatment, the route of administration, and the size, weight, age and overall health of the patient, as is within the skill of one in the art such as a medical practitioner.

The amount of a compound disclosed herein which is administered as a unit dose will depend upon the type of pharmaceutical composition being administered, for example, a solution, a suspension, a gel, a film, an emulsion, a powder, or a sustained-release formulation. In some examples, the effective dosage will be lower than dose amounts needed for oral, intravenous, intramuscular or subcutaneous administration, since transmucosal or transdermal delivery may allow for a more concentrated level of a compound disclosed herein within the facial and head region. The quantity of formulation needed to deliver the desired dose will also depend on the concentration of the compound of the disclosure in the composition. Such determinations are within the skill of one in the art.

The therapeutic dosage of a compound disclosed herein in the pharmaceutical compositions used in the methods of the disclosure will depend on a number of factors such as the chemical composition and/or modification of the compound, its bioavailability by the chosen route of administration, its efficacy, the desired frequency of administration combined with the desired single dosage of the formulation and whether the compound is administered in combination with other active agent(s). Particularly, the dosage of a compound disclosed herein will be chosen to maximize cognitive functions of a subject. Pharmacological data can be obtained from animal models and clinical trials with normal human volunteers or patients by one with skill in the art.

As stated above, an effective amount of a compound (e.g., oxytocin) of the disclosure will depend on the form and composition being used in the method. For example, dosages used for administration of a compound disclosed herein can include, but are not limited to, an effective amount within the dosage range of about 0.1 IU to about 150 IU, or within 1 IU to about 100 IU, or within 10 IU to about 100 IU, or within about 25 IU to about 50 IU, or within about 1 IU to about 40 IU, or within about 1 IU to about 30 IU, or within about 4 IU to about 16 IU, or within about 4 IU to about 24 IU.

Dosages can be administered in a single dose or in multiple doses, for example, dosages can be administered two, three, four, up to ten times daily depending on the type of treatment as well as on individual susceptibility. Dosages can be administered in a sustained release formulation which may allow for a compound disclosed herein to be administered less frequently such as six times a week, five times a week, four times a week, three times a week, twice a week, or once a week, once a month, once every two months, three months, four months, five months or six months or more. Infrequent administration can be accomplished by sustained release formulations.

In some embodiments of the disclosure, a composition comprising a compound of the disclosure may further comprise an additional active agent, wherein the compound and the additional active agent(s) are administered as a mixture, separately and simultaneously, or separately in any order. In some examples the composition comprising a compound disclosed herein is administered in combination with at least one additional active agent. In other examples, the composition comprising a compound of the disclosure is administered in combination with at least two additional active agents. In a particular embodiment, a composition comprising a compound disclosed herein can also be administered in combination with other classes of compounds, including, but not limited to, sepsis treatments, such as drotrecogin-α; steroidals, such as hydrocortisone; local or general anesthetics, such as ketamine; platelet aggregation inhibitors, such as clopidogrel; HMG-CoA reductase inhibitors (statins), such as atorvastatin; anticoagulants, such as heparin; thrombolytics, such as streptokinase; fibrates, such as clofibrate; bile acid sequestrants, such as colestipol; non-steroidal anti-inflammatory agents (NSAIDs), such as naproxen; cholesteryl ester transfer protein (CETP) inhibitors, such as anacetrapib; anti-bacterial agents, such as ampicillin; anti-fungal agents, such as amorolfine; norepinephrine reuptake inhibitors (NRIs), such as atomoxetine; dopamine reuptake inhibitors (DARIs), such as methylphenidate; sedatives, such as diazepham; norepinephrine-dopamine reuptake inhibitor (NDRIs), such as bupropion; serotonin-norepinephrine-dopamine-reuptake-inhibitors (SNDRIs), such as venlafaxine; monoamine oxidase inhibitors, such as selegiline; hypothalamic phospholipids; endothelin converting enzyme (ECE) inhibitors, such as phosphoramidon; opioids, such as tramadol; thromboxane receptor antagonists, such as ifetroban; potassium channel openers; thrombin inhibitors, such as hirudin; hypothalamic phospholipids; growth factor inhibitors, such as modulators of PDGF activity; platelet activating factor (PAF) antagonists; anti-platelet agents, such as GPIIb/IIIa blockers (e.g., abdximab, eptifibatide, and tirofiban), P2Y(AC) antagonists (e.g., clopidogrel, ticlopidine and CS-747), and aspirin; low molecular weight heparins, such as enoxaparin; Factor VIIa Inhibitors and Factor Xa Inhibitors; renin inhibitors; neutral endopeptidase (NEP) inhibitors; vasopepsidase inhibitors (dual NEP-ACE inhibitors), such as omapatrilat and gemopatrilat; squalene synthetase inhibitors; fibrates; niacin; anti-atherosclerotic agents, such as ACAT inhibitors; MTP Inhibitors; calcium channel blockers, such as amlodipine besylate; potassium channel activators; alpha-muscarinic agents; beta-muscarinic agents, such as carvedilol and metoprolol; antiarrhythmic agents; diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, tricrynafen, chlorthalidone, furosenilde, musolimine, bumetanide, triamterene, amiloride, and spironolactone; recombinant tPA, streptokinase, urokinase, prourokinase, and anisoylated plasminogen streptokinase activator complex (APSAC); anti-diabetic agents, such as biguanides (e.g. metformin), glucosidase inhibitors (e.g., acarbose), insulins, meglitinides (e.g., repaglinide), sulfonylureas (e.g., glimepiride, glyburide, and glipizide), thiozolidinediones (e.g. troglitazone, rosiglitazone and pioglitazone), and PPAR-gamma agonists; mineralocorticoid receptor antagonists, such as spironolactone and eplerenone; growth hormone secretagogues; aP2 inhibitors; phosphodiesterase inhibitors, such as PDE III inhibitors (e.g., cilostazol) and PDE V inhibitors (e.g., sildenafil, tadalafil, vardenafil); protein tyrosine kinase inhibitors; antiinflammatories; antiproliferatives, such as methotrexate, FK506 (tacrolimus, Prograf), mycophenolate mofetil; chemotherapeutic agents; immunosuppressants; anticancer agents and cytotoxic agents (e.g., alkylating agents, such as nitrogen mustards, alkyl sulfonates, nitrosoureas, ethylenimines, and triazenes); antimetabolites, such as folate antagonists, purine analogues, and pyridine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids (e.g., cortisone), estrogens/antiestrogens, androgens/antiandrogens, progestins, and luteinizing hormone-releasing hormone anatagonists, and octreotide acetate; microtubule-disruptor agents, such as ecteinascidins; microtubule-stabilizing agents, such as pacitaxel, docetaxel, and epothilones A-F; plant-derived products, such as vinca alkaloids, epipodophyllotoxins, and taxanes; and topoisomerase inhibitors; prenyl-protein transferase inhibitors; and cyclosporins; steroids, such as prednisone and dexamethasone; cytotoxic drugs, such as azathiprine and cyclophosphamide; TNF-alpha inhibitors, such as tenidap; anti-TNF antibodies or soluble TNF receptor, such as etanercept, rapamycin, and leflunomide; and cyclooxygenase-2 (COX-2) inhibitors, such as celecoxib and rofecoxib; and miscellaneous agents such as, hydroxyurea, procarbazine, mitotane, hexamethylmelamine, gold compounds, platinum coordination complexes, such as cisplatin, satraplatin, and carboplatin.

In another embodiment, a composition comprising a compound of the disclosure can be administered with an antipsychotic. Examples of antipsychotics include, but are not limited to, chlorpromazine, fluphenazine haloperidol, perphenazine, aripiprazole, asenapine, brexpiprazole, cariprazine, clozapine, iloperidone, lurasidone, olanzapine, paliperidone, quetiapine, risperidone, and ziprasidone.

In yet another embodiment, a composition comprising a compound of the disclosure can be administered with a stimulant. Examples of stimulants include, but are not limited to, dextroamphetamine, methylphenidate, lisdexamfetamine, amphetamine, and methamphetamine.

In another embodiment, a composition comprising a compound of the disclosure can be administered with a antidementia agent. Examples of antidementia agents include, but are not limited to, cholinesterase inhibitors, such as tacrine, donepezil, rivastigmine, galantamine, and ipidacrine; and other anti-dementia drugs, such as ginkgo follium, and memantine.

In a particular embodiment, a composition comprising a compound disclosed herein can be administered prior to or in conjunction with programs to improve cognitive functions, such as executive function. Examples of such programs include mindfulness meditation, multiple classification and ambiguous language activities, aerobic exercise, silent expression of conscious thoughts to oneself in coherent linguistic form, cognitive stimulation programs, working memory training, video games, and Multi-Attribute Task Battery tests.

In one embodiment, the disclosure allows for the treatment of patients with impaired cognitive function (e.g., impaired executive function). In another embodiment, the disclosure allows for preventing the loss of cognitive function in a normal subject, such as an elderly subject, by the prophylactic administration of one or more compounds of the disclosure.

As used herein, the term “cognitive function improving amount”, or “effective amount” means the amount of a compound or a composition comprising a compound disclosed herein that is useful for causing an improvement in cognitive function of a subject prior to receiving an effective amount of a compound or composition disclosed herein.

The following example is provided in illustration of the disclosure and should not be construed in any way as constituting a limitation thereof.

### Examples

Measuring Executive Function Using an Animal Model.

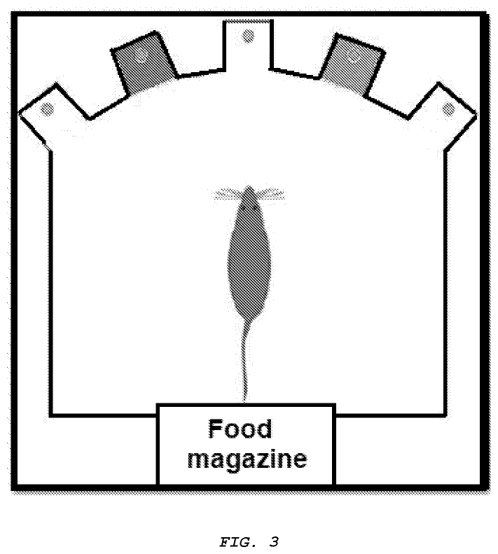

Rats (Brown Norway rats or Long Evans rats) executive function was tested using a nine-choice operant box (e.g., see FIG. 2). The nine-choice operant box has nine depressions in which a rat can poke its nose into (see FIG. 2), where each depression comprises a light emitting diode (LED). A rat was placed in the box and two of the LEDs were illuminated (e.g., see FIG. 3). One LED, the ‘target’, rewarded a rat's nose poke 80% of the time with food while the other LED, the ‘nontarget’, rewarded a rat's nose poke only 20% of the time. After eight consecutive responses in which the ‘target’ LED was selected, the reward ratios for the two LED's were switched, and the next block of testing was begun. Brown Norway rats were used as test subjects while Long Evans rats were used as controls. Brown Norway rats were used as test subjects as they exhibit several features associated with schizophrenia.

Scoring of executive function was based on the number of switches a rat was able to complete within a one-hour test period, as well as the number of block changes a rat can progress through. In particular, the scoring and conclusions of executive function were made as follows:

Switches:

A primary measure of executive function.
The number of times the ‘target’ stimulus switches following eight consecutive “correct” responses by the subject.
The number of switches a rat is able to complete within a one-hour test period indicates how quickly the rat is able to detect and adjust to reversals.
The more switches completed by the rat, the better the executive function of the rat.

Total Trials:

A primary measure of executive function.
Indicates how quickly rat was able to progress through the blocks—the block changes whenever the target switches, so if the subject completed five blocks and is working on the sixth when the one hour testing session ends.
The fewer total trials a rat has, the quicker it progresses through the blocks, and the better its overall performance.

Premature Responses:

A secondary measure of executive function.
Indicates impulsivity.

Testing the Effects of Oxytocin on Executive Function Using an Animal Model.

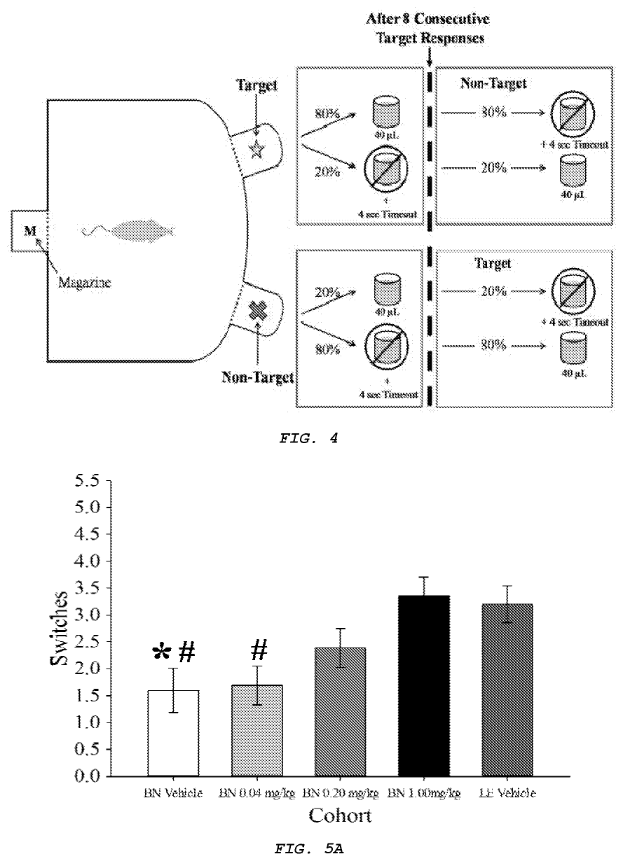

Using the animal model described above, the effects of oxytocin on cognitive flexibly were tested. It was found that number of switches increased when oxytocin was administered to Brown Norway rats, indicating an improvement in executive function, (e.g., see FIG. 4). Moreover, it was further found that when oxytocin was administered at a dose of 1.00 mg/kg the improvement in executive function was significant, and that the improvement of executive function was seen after 7 days post treatment, indicating that oxytocin exerts a long term effect in improving executive function.

Measuring Executive Function Flexibility in Humans.

There are many different tests and protocols utilized to measure executive function. The ability to rapidly switch between focusing on one specific aspect of an object such as “color” to another aspect such as “shape” determines how cognitively flexible you are. Many measures of executive function are regarded as age-specific due to the fact that they are either too simplistic or advanced for other age groups.

A-not-B Task.

This is an executive function test typically administered to children. During this test, children are visually presented with an object that is hidden at “Location A.” The children are then allowed to look for the object at the hidden “Location A”—which is generally within arm's reach. The hiding of the object at Location A is repeated a few times until the child becomes focused on how to find it. Next, the same object is hidden in a new area called “Location B”—a distinct location separate from Location A (also within arm's reach). Individuals under 1 years of age will typically look again in Location A. Children over 1 years of age are able to display “executive function” and learn to find the object at the novel Location B. This is an executive function task reserved for infants and would be far too simplistic for older children, teens, and adults.

Multiple Classification Card Sorting Task.

The Multiple Classification Card Sorting Task is an executive function test administered to children. During the test, various cards are shown to a child, and the child is instructed to sort them based on multiple characteristics (e.g. color and shape). The child will then place them into 4 distinct piles based on the characteristics: red squares, red triangles, purple squares, purple triangles. Studies have suggested that this is a challenging task for most children, but becomes easier once a child reaches age 11.

Evidence highlights that 7 year olds have a difficult time sorting the cards based on multiple characteristics. This suggests that up to a certain age, children are unable to simultaneously focus on two characteristics of a single object. There appears to be a significant jump in executive function between age 7 and 11.

Wisconsin Card Sorting Test (WCST).

Another popular test to measure executive function is the Wisconsin Card Sorting Test (WCST). This test focuses on elements of abstract reasoning and incorporates problem-solving. During the WCST, an individual is presented with cards with displays that differ in elements of: color, shapes, and quantities.

The individual is then handed another set of cards and instructed to match the new cards with the already-presented cards. This is another test administered primarily to children. Individuals between ages 9 and 11 typically have developed enough executive function to properly complete the test.

Stroop Test (Color-Word Naming Test).

In the Stoop Test, an individual is presented with 3 different types of cards: a color card, a word card, and a combo “color-word” card. Their goal is to identify the colors on the color card, the words on the word card, and then solely the colors on the “color-word” card. This is an executive function test primarily reserved for individuals over the age of 11.

### Color Card:

Displays segments of various colors. Individuals are instructed to identify these colors as rapidly as possible.

### Word Card:

Displays the names of colors printed in black and white. Individuals are instructed to read the names of these colors as rapidly as possible.

### Color-Word Card:

Displays the names of colors printed in the ink of an entirely distinct color. In other words, the color of a word does not match up with the text. For example, a person may see the word “Black” with all letters printed in purple ink. When a person sees the Color-Word card, they are instructed to name only the color of the ink, while selectively ignoring the text. In the example of seeing “Black” printed in purple ink, a person would therefore respond with “purple” as quickly as possible.

Delis-Kaplan Executive Function System (D-KEFS).

D-KEFS is a neuropsychological test that is used to measure a variety of verbal and nonverbal executive functions for both children and adults (ages 8-89 years). The D-KEPFS comprises 9 subtests:

The Trail Making Test: measures flexibility of thinking on a visual-motor sequencing task
The Verbal Fluency Test: measures letter fluency, category fluency, and category switching
The Design Fluency Test: measures one's initiation of problem-solving behavior, fluency in generating visual patterns, creativity in drawing new designs, simultaneous processing in drawing the designs while observing the rules and restrictions of the task, and inhibiting previously drawn responses
The Color-Word Interference Test: measures ability to inhibit a dominant and automatic verbal response
The Sorting Test: measures concept-formation skills, modality-specific problem-solving skills (verbal/nonverbal), and the ability to explain sorting concepts abstractly
The Twenty Questions Test: measures the ability to categorize, formulate abstract, yes/no questions, and incorporate the examiner's feedback to formulate more efficient yes/no questions
The Word Context Test: measures verbal modality, deductive reasoning, integration of multiple bits of information, hypothesis testing, and flexibility of thinking
The Tower Test: measures spatial planning, rule learning, inhibition of impulsive and preservative responding, and the ability to establish and maintain instructional set
The Proverb Test: measures one's ability to form novel, verbal abstractions
These 9 subtests generate 16 main achievement scores and hundreds of optional errors, contrast, accuracy, and time-interval scores. As such, use of the computerized scoring assistant makes scoring the measure less time consuming. The assessment is normed with a representative sample. The D-KEFS offers a comprehensive portrayal of individual's executive function skills, and the complexity of these tasks make them sensitive to the detection of even mild brain damage.

Neural Mechanisms of Executive Function.

To determine the specific regions of the brain associated with executive function, fMRI (functional magnetic resonance imaging) scans can be used. The fMRI scans revealed that a variety of cortical networks are activated to facilitate executive function. These networks include: the anterior cingulate cortex, basal ganglia, posterior parietal cortex, and the prefrontal cortex.

The prefrontal cortex is believed to be the driving force behind executive function. The specific regions within the prefrontal cortex that are activated during an executive function task are likely task-dependent. The basal ganglia is known to become active during selection of responses, whereas the posterior parietal cortex is active when preexisting information is updated.

The examples set forth above are provided to give those of ordinary skill in the art a complete disclosure and description of how to make and use the embodiments of the methods, treatments and compositions of the disclosure, and are not intended to limit the scope of what the inventors regard as their disclosure. Modifications of the above-described modes for carrying out the disclosure that are obvious to persons of skill in the art are intended to be within the scope of the following claims.

1. A method of treating a subject that has an impairment in executive function or prophylactically preventing an impairment in executive function of a subject comprising, administering to the subject an effective amount of an oxytocin peptide or analog or a pharmaceutical composition comprising an effective amount of an oxytocin peptide or analog.

2. The method of claim 1, wherein the subject that has an impairment in executive function has a cognitive disorder selected from the group consisting of developmental disorder, aphasia, delirium, dementia, amnesia, executive dysfunction and cerebrovascular disease.

3. The method of claim 2, wherein the developmental disorder is selected from the group consisting of attention-deficit hyperactivity disorder, autism spectrum disorder, and Asperger's disorder.

4. The method of claim 2, wherein the dementia is selected from the group consisting of Alzheimer's disease, cortical dementia, and subcortical dementia.

5. The method of claim 1, wherein the subject has an impairment in executive function has a mental disorder selected from the group consisting of Tourette's syndrome, obsessive-compulsive disorder, unipolar and bipolar affective disorder, Schizotypal personality disorder, corpus callosum dysgenesis, impulsive personality disorder, acute stress disorder, and post-traumatic stress syndrome.

6. The method of claim 1, wherein the subject has an impairment in executive function has a disorder selected from the group consisting of Systemic Lupus Erythematosus, Parkinson's disease, rapid eye movement sleep behavior disorder, and Huntington's disease.

7. The method of claim 1, wherein the subject is a person of at least 60 years in age, wherein the subject may or may not have an impairment in executive function, and wherein the subject does not have a cognitive or mental disorder.

8. The method of claim 7, wherein the subject is a person of at least 65 years in age and has an impairment in executive function.

9. The method of claim 1, wherein the impairment of executive function is a mild impairment of executive function.

10. The method of claim 1, wherein the impairment of executive function is an impairment of executive function.

11. The method of claim 1, wherein the oxytocin peptide or the pharmaceutical composition comprising the oxytocin peptide is administered intranasally, intramuscularly, or intravenously.

12. The method of claim 11, wherein the oxytocin peptide or the pharmaceutical composition comprising the oxytocin peptide is administered intranasally.

13. The method of claim 1, wherein the oxytocin peptide is administered at a dose of about 40-100 IU per day.

14. The method of claim 13, wherein the dose is at least two times per day at a dose of about 20-40 IU per administration.

15. The method of claim 13, wherein the dose is about 40-80 IU per day.

16. The method of claim 1, wherein the oxytocin peptide is administered with at least one additional active agent, and wherein the at least one additional active agent is used to treat a mental or cognitive disorder.

17. The method of claim 16, wherein the at least one additional active agent is an antipsychotic agent, a stimulant, and/or antidementia agent.

18. The method of claim 1, wherein the oxytocin peptide is administered prior to or in conjunction with one or more programs to improve cognitive functions.

19. The method of claim 18, wherein the one or more programs to improve cognitive functions is selected from mindfulness meditation, multiple classification and ambiguous language activities, aerobic exercise, silent expression of conscious thoughts to oneself in coherent linguistic form, cognitive stimulation programs, working memory training, video games, and Multi-Attribute Task Battery tests.


SEQUENCE LISTING (1 sequence)

SEQ ID NO: 1 - protein, 9 aa, Homo sapiens
CYIQNCPLG